(19) United States
(12) Patent Application Publication
(10) Pub. No.: US 2022/0157412 A1
(43) Pub. Date: May 19, 2022

(54) CLINICAL TRIAL MATCHING APPARATUS

(71) Applicants: National University Corporation Tokyo Medical and Dental University, Tokyo (JP); CANON MEDICAL SYSTEMS CORPORATION, Tochigi (JP)

(72) Inventors: Sadakatsu IKEDA, Bunkyo (JP); Eri ISHIBASHI, Meguro (JP); Kei MORI, Shioya-gun (JP); Fuminori FUJITA, Nasushiobara (JP); Shoichi KANAYAMA, Otawara (JP); Kimiko YOKOTA, Shinagawa (JP); Naoki OOWAKI, Nasushiobara (JP)

(73) Assignees: National University Corporation Tokyo Medical and Dental University, Tokyo (JP); CANON MEDICAL SYSTEMS CORPORATION, Tochigi (JP)

(21) Appl. No.: 17/588,399

(22) Filed: Jan. 31, 2022

Related U.S. Application Data
(63) Continuation of application No. PCT/JP2021/001310, filed on Jan. 15, 2021.

(30) Foreign Application Priority Data
Jan. 15, 2020 (JP) 2020-004131

(51) Int. Cl. G16H 10/20; G16H 10/60; G16H 50/70; G16H 70/40; G16H 20/10; G16H 40/20; G16B 40/00

(52) U.S. Cl. CPC G16H 10/20; G16H 10/60; G16H 50/70; G16B 40/00; G16H 20/10; G16H 40/20; G16H 70/40

(57) ABSTRACT

A clinical trial matching apparatus according to an embodiment includes processing circuitry. The processing circuitry receives patient information. The processing circuitry retrieves a clinical trial matching the patient from clinical trials based on the patient information, by referring to storage circuitry that stores data for associating a clinical trial in which medication conditions are set with the results of the clinical trial, for the clinical trials. The processing circuitry presents the retrieved clinical trial. The processing circuitry collects, and stores in the storage circuitry, the results obtained by conducting the retrieved clinical trial on the patient and patient's clinical information. The processing circuitry updates the conditions of the retrieved clinical trial based on the clinical information, when there is clinical information that can identify a significant difference in drug efficacy in the collected clinical information.

### CROSS-REFERENCE TO RELATED APPLICATIONS

This application is a continuation of PCT international application Ser. No. PCT/JP2021/001310 filed on Jan. 15, 2021 which designates the United States, incorporated herein by reference, and which claims the benefit of priority from Japanese Patent Applications No. 2020-004131, filed on Jan. 15, 2020, incorporated herein by reference.

### FIELD

Embodiments described herein relate generally to a clinical trial matching apparatus.

### BACKGROUND

Cancer genomic medicine is a medical treatment that treats a patient according to the constitution and symptoms of the patient and the like, by examining a large number of genes mainly in cancer tissues and revealing gene mutations. For example, in hospitals, a therapeutic agent suitable for the patient is determined, by confirming the gene mutation information in the patient and discussing the treatment strategy. When there is no therapeutic agent suitable for the patient, a clinical trial will be therapeutic options.

The clinical trial is a clinical study carried out to obtain approval of a new drug developed by a pharmaceutical company from governmental institutions (for example, Ministry of Health, Labor and Welfare) as “medicine”. In general, clinical trials are conducted in medical institutions such as a hospital in response to a request from a pharmaceutical company, and by administering a new drug to a subject such as a patient. There is also doctor-initiated clinical trials in which a new drug or a new treatment is evaluated under the leadership of a medical institution or a doctor. The pharmaceutical company confirms the safety, efficacy, usage, dosage, and the like of a new drug on the basis of the results of the clinical trial. When the results of the clinical trial are favorable, the new drug is approved as a “pharmaceutical product”.

### BRIEF DESCRIPTION OF THE DRAWINGS

FIG. 1 is a diagram illustrating an example of a configuration of a clinical trial matching system including a clinical trial matching apparatus according to the present embodiment;

FIG. 2 is a diagram illustrating an example of a patient information table stored in storage circuitry;

FIG. 3 is a diagram illustrating an example of a clinical trial information table stored in the storage circuitry;

FIG. 4 is a diagram illustrating an example of conditions included in clinical trial information;

FIG. 5 is a diagram illustrating an example of a reception screen;

FIG. 6 is a diagram illustrating an example of a retrieval result screen;

FIG. 7 is a diagram for explaining a process executed by an update function;

FIG. 8 is a diagram for explaining a process executed by the update function;

FIG. 9 is a diagram illustrating an example of the reception screen;

FIG. 10 is a diagram illustrating an example of the retrieval result screen; and

FIG. 11 is a flowchart illustrating a procedure of a process performed by the clinical trial matching system including the clinical trial matching apparatus according to the present embodiment.

### DETAILED DESCRIPTION

A clinical trial matching apparatus according to an embodiment includes processing circuitry. The processing circuitry receives patient information. The processing circuitry retrieves a clinical trial matching the patient from clinical trials based on the patient information, by referring to storage circuitry that stores data for associating a clinical trial in which medication conditions are set with the results of the clinical trial, for the clinical trials. The processing circuitry presents the retrieved clinical trial. The processing circuitry collects, and stores in the storage unit, the results obtained by conducting the retrieved clinical trial on the patient and patient's clinical information. The processing circuitry updates the conditions of the retrieved clinical trial based on the clinical information, when there is clinical information that can identify a significant difference in drug efficacy in the collected clinical information.

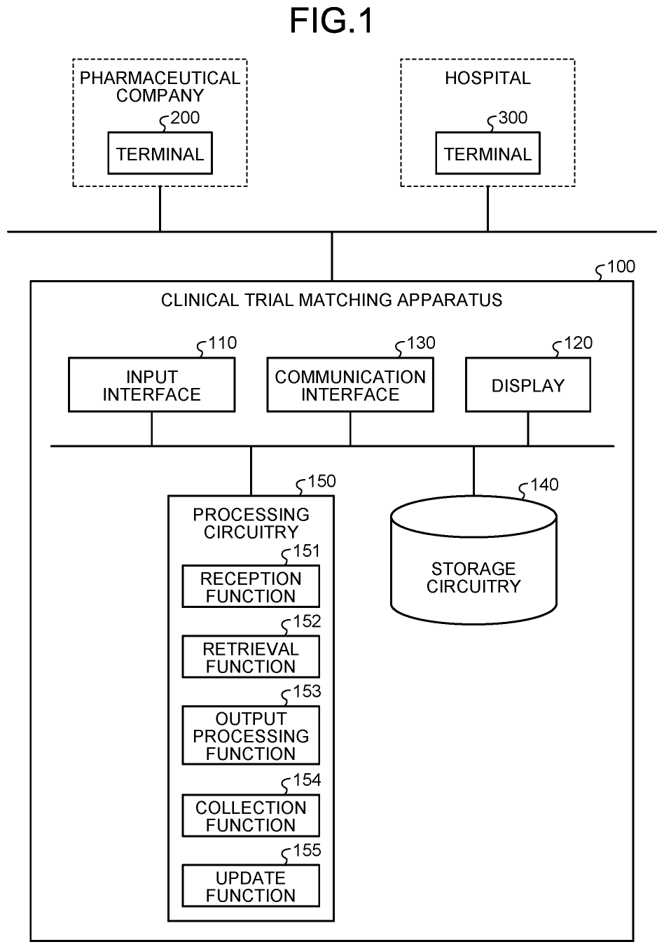

Hereinafter, an embodiment of a clinical trial matching apparatus will be described in detail with reference to the accompanying drawings. Hereinafter, a clinical trial matching system including a clinical trial matching apparatus will be described as an example. In the clinical trial matching system illustrated in FIG. 1, one of the devices is illustrated. However, in practice, the clinical trial matching system may also include a plurality of devices.

FIG. 1 is a diagram illustrating an example of a configuration of a clinical trial matching system 1 including a clinical trial matching apparatus 100 according to the present embodiment. The clinical trial matching system 1 illustrated in FIG. 1 includes the clinical trial matching apparatus 100, a terminal 200, and a terminal 300.

For example, the terminal 200 is a device installed in a pharmaceutical company, and is operated by a staff of the pharmaceutical company. When the pharmaceutical company has developed a new drug, a clinical trial, which is a clinical study of the new drug, needs to be conducted by a medical institution such as a hospital (hereinafter, simply referred to as a “hospital”). In this case, the staff of the pharmaceutical company operates the terminal 300, and uploads the clinical trial information of the clinical trial on the clinical trial matching apparatus 100. The clinical trial information will be described below.

For example, the terminal 300 is a device installed in a hospital, and is operated by a doctor or a staff such as a person in charge of the examination. The terminal 300 may also be a hospital information system (HIS) server that manages information generated in the hospital. For example, in hospitals, a therapeutic agent suitable for the patient is determined, by confirming the gene mutation information in the patient, and discussing the treatment strategy. When there is no therapeutic agent suitable for the patient, clinical trials will be therapeutic options. In this example, a drug that has already been approved is referred to as a “therapeutic agent”, and a drug to be investigated is referred to as an “investigational drug”, which will be described below.

More specifically, when there is no therapeutic agent suitable for the patient, the doctor determines the treatment strategy with the patient, and obtains the consent of the patient to participate in a clinical trial. If the consent is obtained, the clinical trial is conducted. The clinical trial will be conducted in the hospital, or in a hospital where the patient is transferred on referral from the doctor. For example, upon obtaining the consent of the patient to participate in the clinical trial, the doctor operates the terminal 300 and uploads information on the patient (hereinafter, referred to as “patient information”) to the clinical trial matching apparatus 100. In this example, the patient information will be described below.

The clinical trial matching apparatus 100 is communicably connected to the terminal 200 and the terminal 300 via a network. For example, the clinical trial matching apparatus 100 is implemented by a computer apparatus such as a work station and a personal computer.

The clinical trial matching apparatus 100 includes an input interface 110, a display 120, a communication interface 130, storage circuitry 140, and processing circuitry 150. The configuration of the clinical trial matching apparatus 100 is not limited to the configuration described above. For example, the clinical trial matching apparatus 100 may only include the processing circuitry 150, and the input interface 110, the display 120, and the communication interface 130 may be used by being connected to the clinical trial matching apparatus 100.

The input interface 110 is connected to the processing circuitry 150, and receives input operations of various instructions and various types of information from an operator. More specifically, the input interface 110 converts the input operation received from the operator to an electrical signal, and outputs the electrical signal to the processing circuitry 150. For example, the input interface 110 is implemented by a trackball, a switch button, a mouse, a keyboard, a touch pad that performs an input operation by touching the operation surface, a touch screen in which a display screen and a touch pad are integrated, a non-contact input circuit using an optical sensor, an audio input circuit, and the like. In the present embodiment, the input interface 110 is not limited to one including physical operation parts such as a mouse and a keyboard. For example, the input interface 110 may also include an electrical signal processing circuitry that receives an electrical signal corresponding to an input operation from an external input device provided separately from the device, and that outputs the electrical signal to the control circuit.

The display 120 is connected to the processing circuitry 150, and displays various types of information and various images. More specifically, the display 120 converts data on various types of information and various images sent from the processing circuitry 150 into electrical signals for display, and outputs the electrical signals. For example, the display 120 is implemented by a liquid crystal monitor, a cathode ray tube (CRT) monitor, a touch panel, and the like.

The communication interface 130 is connected to the processing circuitry 150, and controls the transmission and communication of various types of data performed between the clinical trial matching apparatus 100 and each system. For example, the communication interface 130 controls the transmission and communication of various types of data performed between the clinical trial matching apparatus 100, the terminal 200, and the terminal 300. For example, the communication interface 130 is implemented by a network card, a network adaptor, a network interface controller (NIC), and the like.

The storage circuitry 140 is connected to the processing circuitry 150, and stores therein various types of information. More specifically, the storage circuitry 140 stores therein patient information received from various systems. For example, the storage circuitry 140 is implemented by a random access memory (RAM), a semiconductor memory element such as a flash memory, a hard disk, an optical disc, or the like. If the clinical trial matching apparatus 100 is accessible on the network, the storage circuitry 140 may not be built in the clinical trial matching apparatus 100. In this example, the storage circuitry 140 is an example of a storage unit.

The processing circuitry 150 controls the components of the clinical trial matching apparatus 100. For example, the processing circuitry 150 executes a reception function 151, a retrieval function 152, an output processing function 153, a collection function 154, and an update function 155. In this example, the processing functions executed by the reception function 151, the retrieval function 152, the output processing function 153, the collection function 154, and the update function 155 that are the components of the processing circuitry 150 are stored in the storage circuitry 140 in the form of computer-executable programs, for example. The processing circuitry 150 is a processor that implements the function corresponding to each computer program, by reading out each computer program from the storage circuitry 140, and executing the computer program. In other words, the processing circuitry 150 that has read out the computer programs has the functions illustrated in the processing circuitry 150 in FIG. 1. In this example, the reception function 151 is an example of a reception unit. The retrieval function 152 is an example of a retrieval unit. The output processing function 153 is an example of a presentation unit. The collection function 154 is an example of a collection unit. The update function 155 is an example of an update unit.

For example, the term “processor” explained above indicates a circuit such as a central processing unit (CPU), a graphics processing unit (GPU), an application specific integrated circuit (ASIC), and a programmable logic device (for example, a simple programmable logic device (SPLD), a complex programmable logic device (CPLD), and a field programmable gate array (FPGA)). For example, when the processor is a CPU, the processor implements the function by reading and executing the computer program stored in the storage circuitry. For example, when the processor is an ASIC, the computer program is built directly into the circuit of the processor, instead of being stored in the storage circuitry. The processors in the present embodiment do not each necessarily have to be structured as a single circuit. It is also possible to structure one processor by combining a plurality of independent circuits to implement the functions. The components illustrated in FIG. 1 may also be integrated into one processor to implement the functions.

The overall configuration of the clinical trial matching system 1 including the clinical trial matching apparatus 100 according to the present embodiment has been described. Under such a configuration, for example, the clinical trial matching apparatus 100 is used to find a clinical trial suitable for the patient.

For example, a clinical trial is conducted in the hospital by administering a new drug (hereinafter, referred to as an “investigational drug”) to a subject such as a patient, and the pharmaceutical company confirms the safety, efficacy, usage, dosage, and the like of the investigational drug on the basis of the results of the clinical trial. In this process, when the results of the clinical trial are favorable, the investigational drug is approved as a “pharmaceutical product”. However, it requires a lot of time and cost to approve an investigational drug as a “pharmaceutical product”. Thus, efficient clinical trials are in great demand in pharmaceutical companies.

Hence, the clinical trial matching apparatus 100 according to the present embodiment performs the following process to improve the results of clinical trials. First, the reception function 151 receives information on the patient (patient information). The retrieval function 152 retrieves the clinical trial that matches the patient from a plurality of clinical trials on the basis of the patient information, by referring to the storage circuitry 140 that stores therein data for associating a clinical trial in which medication conditions are set with the results of the clinical trial, for the clinical trials. The output processing function 153 presents information on the retrieved clinical trial. The collection function 154 collects a clinical trial results obtained by conducting the retrieved clinical trial on the patient and the clinical information on the patient. The collection function 154 then stores the results and information in the storage circuitry 140. When there is clinical information that can identify a significant difference in drug efficacy in the collected clinical information, the update function 155 updates the above-described conditions of the retrieved clinical trial, on the basis of the clinical information.

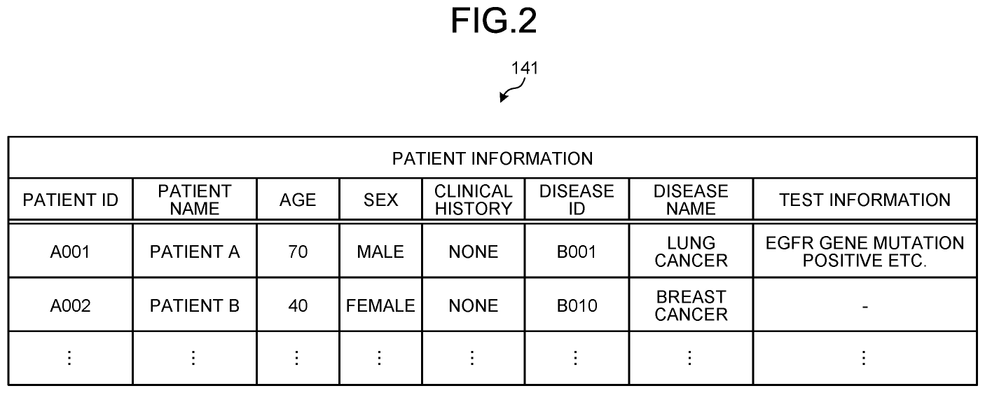

Next, information stored in the storage circuitry 140 will be described. FIG. 2 is a diagram illustrating an example of a patient information table 141 stored in the storage circuitry 140. The patient information table 141 stores therein a plurality of pieces of patient information. The patient information is information uploaded to the clinical trial matching apparatus 100 from the terminal 300 in the hospital. The patient information is information such as an electronic medical record created for a patient, and for example, includes information on the examination, diagnosis, and the like.

More specifically, the patient information includes a patient identification (ID) for identifying the patient, a patient name indicating the name of the patient, age (birth date and year), sex, clinical history, and the like. For example, the patient ID “A001”, the patient name “patient A”, age “70”, sex “male”, and the like are registered in the patient information table 141 as patient information.

The patient information also includes a disease ID for identifying the disease of the patient, a disease name indicating the name of the disease, test information indicating the results of the genetic test of the patient, and the like. For example, the disease ID “B001”, the disease name “lung cancer”, the test information “epidermal growth factor receptor (EGFR) gene mutation positive”, and the like are registered in the patient information table 141 as patient information, in association with the patient ID “A001”. In this example, the test information also includes vital information of the patient and information such as a past medical history, in addition to the results of the genetic test. The vital information includes the results of the patient's blood test, and nursing records such as body temperature, blood pressure, and pulse of the patient. For example, the vital information and the information on the past medical history and the like are obtained from the HIS server as electronic medical record information.

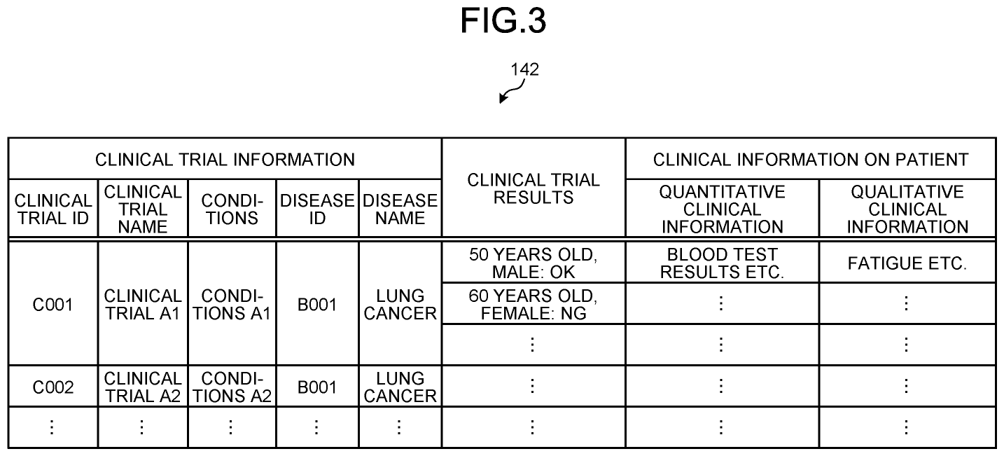

FIG. 3 is a diagram illustrating an example of a clinical trial information table 142 stored in the storage circuitry 140. The clinical trial information table 142 stores therein a plurality of pieces of clinical trial information. The clinical trial information is information uploaded to the clinical trial matching apparatus 100 from the terminal 200 in the pharmaceutical company.

More specifically, the clinical trial information includes a clinical trial ID for identifying the clinical trial, a clinical trial name indicating the name of the clinical trial, conditions for administering the investigational drug in the clinical trial, a disease ID for identifying the disease to be investigated, a disease name indicating the name of the disease, and the like. For example, the clinical trial ID “C001”, the clinical trial name “clinical trial A1”, the conditions “conditions A1” for administering the investigational drug in the “clinical trial A1”, the disease ID “B001”, the disease name “lung cancer”, and the like are registered in the clinical trial information table 142 as clinical trial information. The clinical trial ID “C002”, the clinical trial name “clinical trial A2”, the conditions “conditions A2” for administering the investigational drug in the “clinical trial A2”, the disease ID “B001”, the disease name “lung cancer”, and the like are registered in the clinical trial information table 142 as clinical trial information.

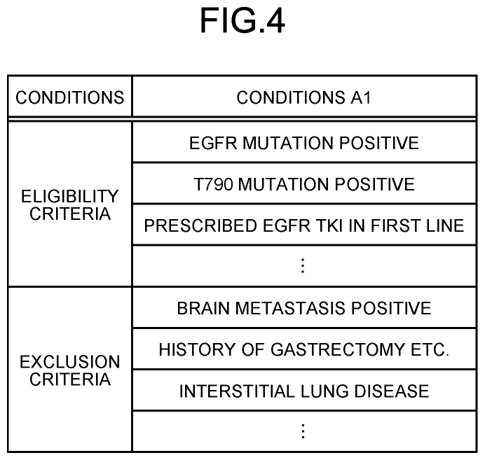

The conditions included in the clinical trial information are set in the clinical trial plan document (hereinafter, referred to as a “clinical trial protocol”). The conditions include “eligibility criteria” for assessing eligibility for the clinical trial, and “exclusion criteria” that do not meet the eligibility criteria. For example, as illustrated in FIG. 4, items such as “EGFR mutation positive”, “T790 mutation positive”, “prescribed EGFR tyrosine kinase inhibitors (EGFR TKIs) in the first line”, and the like, are set in the conditions “conditions A1” included in the clinical trial information with the clinical trial ID “C001”, as the “eligibility criteria”. Items such as “brain metastasis positive”, “history of gastrectomy and the like”, “interstitial lung disease”, and the like are set in the conditions “conditions A1”, as the “exclusion criteria”.

Moreover, as illustrated in FIG. 3, the clinical trial information table 142 stores therein the clinical trial results and the clinical information on the patient, for each of the pieces of clinical trial information. The details of the clinical trial results and the clinical information on the patient will be described below.

Next, a process executed by the reception function 151 and the output processing function 153 will be described. As described above, the reception function 151 receives the patient information, and stores the patient information in the storage circuitry 140.

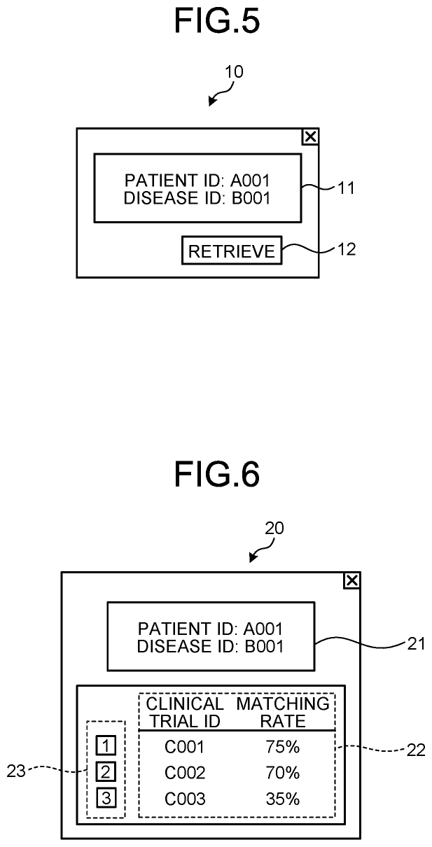

More specifically, the reception function 151 receives the patient information (patient ID, patient name, age, sex, clinical history, disease ID, disease name, test information, and the like) uploaded to the clinical trial matching apparatus 100 from the terminal 300 in the hospital. The reception function 151 then stores the received patient information in the patient information table 141 in the storage circuitry 140. For example, the received patient information is the patient information including the patient ID “A001”. In this case, the output processing function 153 transmits a message indicating that the patient information including the patient ID “A001” is received, to the terminal 300 in the hospital as the reception information. The terminal 300 receives the reception information, and causes the display of the terminal 300 to display a reception screen 10 illustrated in FIG. 5 as the received reception information. For example, the reception screen 10 includes a display field 11 that displays the patient ID “A001” and the disease ID “B001” in the received patient information, and a retrieval button 12 operated by the staff of the hospital to perform a retrieval request, which will be described below.

Next, a process executed by the retrieval function 152 and the output processing function 153 will be described. As described above, the retrieval function 152 retrieves the clinical trial that matches the patient having the received patient information, from a plurality of clinical trials in which conditions for administering the investigational drug are set, by referring to the clinical trial information table 142 in the storage circuitry 140. The output processing function 153 presents information on the retrieved clinical trial.

More specifically, for example, it is assumed that when the reception screen 10 is displayed on the display of the terminal 300 in the hospital, the staff of the hospital operates the retrieval button 12 on the reception screen 10. In this case, the terminal 300 transmits a retrieval request to the clinical trial matching apparatus 100. The retrieval function 152 retrieves the clinical trial information that matches the patient information including the patient ID “A001”, from the pieces of clinical trial information stored in the clinical trial information table 142 in the storage circuitry 140, according to the retrieval request. In this process, the output processing function 153 transmits the retrieved clinical trial information to the terminal 300 in the hospital. The terminal 300 receives the clinical trial information, and causes the display of the terminal 300 to display a retrieval result screen 20 illustrated in FIG. 6, as the received clinical trial information. For example, the retrieval result screen 20 includes a display field 21 that displays the patient ID “A001” and the disease ID “B001”, a display field 22 that displays the retrieved clinical trial information, and a selection button 23 that selects one clinical trial information from the retrieved clinical trial information.

For example, it is assumed that the retrieval function 152 retrieves pieces of clinical trial information including clinical trial IDs “C001”, “C002”, and “C003”, as the pieces of clinical trial information that match with the patient information including the patient ID “A001”. For example, the retrieval function 152 retrieves the pieces of clinical trial information including the clinical trial IDs “C001”, “C002”, and “C003”, on the basis of the age, sex, clinical history, disease name, test information and the like included in the patient information, and the conditions (“eligibility criteria” and “exclusion criteria”) for administering the investigational drug in the clinical trial. In this example, in the clinical trials with the clinical trial IDs “C001” and “C002”, it is assumed that the age, sex, clinical history, disease name, test information, and the like included in the patient information, and the conditions (“eligibility criteria” and “exclusion criteria”) for administering the investigational drug in the clinical trial match with the patient information including the patient ID “A001” at a high probability. In this process, it is assumed that the matching rates of the pieces of the clinical trial information with respect to the clinical trial IDs “C001”, “C002”, and “C003” are “75%”, “70%”, and “35%”, respectively. In this case, in the display of the terminal 300 in the hospital, the pieces of clinical trial information including the clinical trial IDs “C001”, “C002”, and “C003” are displayed on the display field 22 of the retrieval result screen 20, in descending order of the matching rate. In other words, the matching rates of “75%”, “70%”, and “35%” are displayed on the display field 22 with respect to the clinical trial IDs “C001”, “C002”, and “C003”, respectively. Moreover, selection buttons 23 “1”, “2”, and “3” are displayed on the display field 22 in descending order of the matching rate, with respect to the clinical trial IDs “C001”, “C002”, and “C003”, respectively.

As described above, among the clinical trials with the clinical trial IDs “C001”, “C002”, and “C003”, the clinical trial that matches the most with the patient information including the patient ID “A001” is the clinical trial with the clinical trial ID “C001”. Hence, for example, it is assumed that when the retrieval result screen 20 is displayed on the display of the terminal 300 in the hospital, the staff of the hospital operates the selection button 23 “1” on the retrieval result screen 20, and selects the clinical trial information including the clinical trial ID “C001”. In this case, the terminal 300 transmits a message indicating that the clinical trial information including the clinical trial ID “C001” is selected, to the clinical trial matching apparatus 100 as the selection information. The output processing function 153 notifies the patient information such as the patient ID “A001”, the patient name “patient A”, age “70”, and sex “M (male)” to the terminal 200 in the pharmaceutical company that has uploaded the clinical trial information including the clinical trial ID “C001” to the clinical trial matching apparatus 100, according to the selection information. In other words, the output processing function 153 introduces the patient with the patient ID “A001” to the pharmaceutical company as a clinical trial subject. Moreover, in the hospital, the clinical trial with the clinical trial ID “C001” is conducted on the patient with the patient ID “A001”, as the selected clinical trial information.

Next, a process executed by the collection function 154 will be described. As described above, the collection function 154 collects the clinical trial results obtained by conducting the retrieved clinical trial on the patient with the patient ID “A0001”, and the clinical information on the patient. The collection function 154 then stores the results and information in the clinical trial information table 142 in the storage circuitry 140.

More specifically, the clinical trial results and the clinical information on the patient are uploaded from the terminal 300 in the hospital that conducts the clinical trial with the clinical trial ID “C0001”, with the anonymized patient information. In other words, the clinical trial results and the clinical information on the patient are fed back to the clinical trial matching apparatus 100 with the anonymized patient information. The collection function 154 collects the clinical trial results and the clinical information on the patient uploaded from the terminal 300 in the hospital that conducts the clinical trial with the clinical trial ID “C0001”. The collection function 154 then stores the results and information in the clinical trial information table 142 in the storage circuitry 140.

As illustrated in FIG. 3, for example, the clinical trial results are registered in the clinical trial information table 142 in association with the clinical trial ID “C0001”. The clinical trial results include information on one of “OK” indicating that the investigational drug is significantly effective, “NG” indicating that the investigational drug is not effective, and “Soso” indicating neither of the two. An example of the patient whose clinical trial results are “NG” includes a case when an investigational drug in a certain clinical trial is administered to a patient, but strong side effects were observed in the patient, and the clinical trial cannot be continued any longer and had to be stopped and the like. An example of the patient whose clinical trial results are “Soso” includes a case when an investigational drug in a certain clinical trial is administered to a patient, and the efficacy of the investigational drug is observed, but the efficacy does not last, and the like. In this manner, even if the conditions set in the clinical trial protocol are satisfied, the investigational drug may not be effective for some patients, and may be remarkably effective for the other patients. In the present embodiment, by feeding back the clinical trial results to the clinical trial matching apparatus 100, it is possible to more suitably match the patient with the clinical trial.

Moreover, as illustrated in FIG. 3, clinical information on the patient is registered in the clinical trial information table 142 in association with the clinical trial ID “C0001”. For example, clinical information on a patient includes quantitative clinical information that can be quantified such as patient's vital, and qualitative clinical information difficult to quantify. The details of the quantitative clinical information and the qualitative clinical information will be described below.

Next, a process executed by the update function 155 will be described. As described above, when there is clinical information that can identify a significant difference in drug efficacy, in the clinical information on the patient collected by the collection function 154, the update function 155 updates the conditions of the clinical trial retrieved by the retrieval function 152 on the basis of the clinical information.

More specifically, for example, the process executed by the update function 155 is performed when the number of results of the clinical trial with the clinical trial ID “C0001” exceeds a set number. In this example, it is assumed that the number of results of the clinical trial with the clinical trial ID “C0001” exceeds the set number.

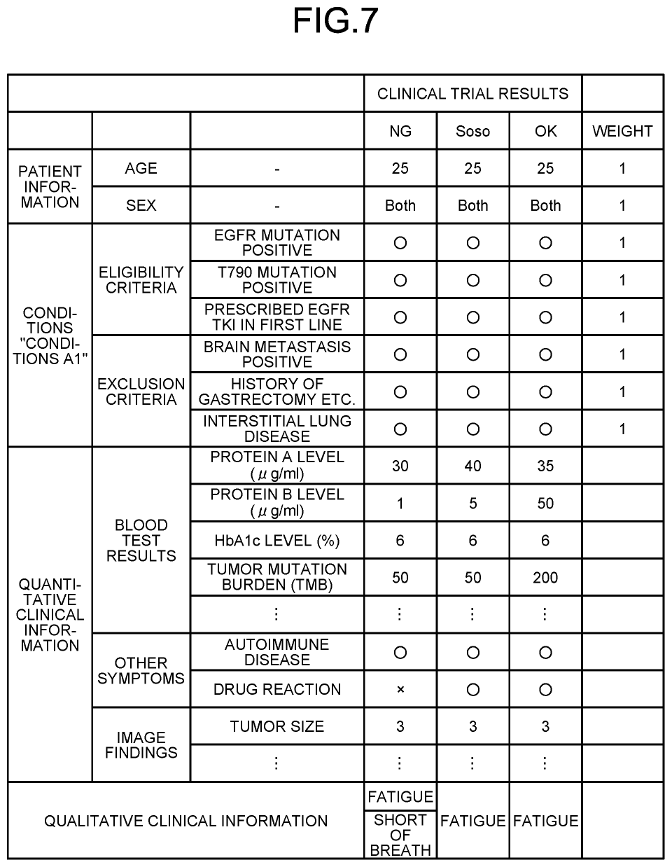

In this case, first, the update function 155 analyzes the results of the clinical trial with the clinical trial ID “C0001” and the clinical information on the patient registered in the clinical trial information table 142. FIG. 7 is a diagram for explaining a process executed by the update function 155. FIG. 7 illustrates a relation among the conditions “conditions A1” set for the clinical trial, and the anonymized patient information, the clinical trial results, and the clinical information on the patient that are information fed back to the clinical trial matching apparatus 100, in the clinical trial with the clinical trial ID “C0001”.

For example, in FIG. 7, the anonymized patient information includes the age, sex, and the like of the patient. In FIG. 7, for the convenience of explanation, the ages are expressed by the average values for the clinical trial results “NG”, “Soso”, and “OK”. However, for example, the ages may also be represented in a distribution map in which the horizontal axis is the age and the vertical axis is the number of patients.

For example, in the conditions “conditions A1” in FIG. 7, items such as “EGFR mutation positive”, “T790 mutation positive”, “prescribed EGFR tyrosine kinase inhibitors (EGFR TKIs) in the first line”, and the like are set as the “eligibility criteria”. Items such as “brain metastasis positive”, “history of gastrectomy and the like”, “interstitial lung disease”, and the like are also set as the “exclusion criteria”.

For example, in FIG. 7, the clinical information on the patient includes information on blood test results as the quantitative clinical information. The blood test results include information on protein, information on blood glucose, information on genes, and the like. The information on protein includes a “protein A level (μg/ml)”, a “protein B level (μg/ml)”, and the like. The information on blood glucose includes an “HbA1c level (%)” and the like. The information on genes includes “tumor mutation burden (TMB)” and the like. In FIG. 7, for the convenience of explanation, the blood test results are expressed by the average values for the clinical trial results “NG”, “Soso”, and “OK”. However, for example, the blood test results may also be represented in a distribution map in which the horizontal axis is the blood test results and the vertical axis is the number of patients.

For example, in FIG. 7, the clinical information on the patient includes information on quality of life (QOL) as the qualitative clinical information. For example, the information on QOL includes symptoms such as “fatigue”, “short of breath”, “have trouble waking up”, “feeling of body ache”, “numbed hand”, “palpitations”, and the like caused by the prescribed drug. More specifically, these symptoms may not be caused by disease, may be subjective, and may occur during a recovery period and the like.

In FIG. 7, the items are weighted. The weight is a coefficient indicating the range between 0 and 1. For example, the weight is used as a condition for administering the investigational drug used in the clinical trial with the clinical trial ID “C0001”. The weight “1” is set for the “eligibility criteria” and the “exclusion criteria” in the conditions “conditions A1”. The weight “1” is also set for the age and sex of the patient. A flag may also be used instead of the weight.

Next, to analyze the results of the clinical trial with the clinical trial ID “C0001” and the clinical information on the patient (quantitative clinical information and qualitative clinical information), the update function 155 determines whether there is quantitative clinical information that can identify a significant difference in drug efficacy, in the quantitative clinical information on the clinical trial with the clinical trial ID “C0001”. In this example, the quantitative clinical information will be described using the blood test results as an example.

For example, in FIG. 7, it is assumed that the “protein A level”, which is quantitative clinical information, is “30” in the patient whose clinical trial results are “NG”, “40” in the patient whose clinical trial results are “Soso”, and “35” in the patient whose clinical trial results are “OK”. In this example, there is no significant difference between the patient whose clinical trial results are “OK” and the other patients. In this case, the update function 155 determines that the “protein A level” is not the quantitative clinical information that can identify a significant difference in drug efficacy.

For example, in FIG. 7, it is assumed that the “protein B level”, which is quantitative clinical information, is “1” in the patient whose clinical trial results are “NG”, “5” in the patient whose clinical trial results are “Soso”, and “50” in the patient whose clinical trial results are “OK”. In this example, there is a difference of 10 times or more between the patient whose clinical trial results are “OK” and the other patients, and it is assumed that a statistically significant difference is also identified. In this case, the update function 155 determines that the “protein B level” is the quantitative clinical information that can identify a significant difference in drug efficacy.

For example, in FIG. 7, it is assumed that the “HbA1c level”, which is quantitative clinical information, is “6” in the patient whose clinical trial results are “NG”, “6” in the patient whose clinical trial results are “Soso”, and “6” in the patient whose clinical trial results are “OK”. In this example, there is no significant difference between the patient whose clinical trial results are “OK” and the other patients. In this case, the update function 155 determines that the “HbA1c level” is not the quantitative clinical information that can identify a significant difference in drug efficacy.

For example, in FIG. 7, it is assumed that the “tumor mutation burden”, which is quantitative clinical information, is “50” in the patient whose clinical trial results are “NG”, “50” in the patient whose clinical trial results are “Soso”, and “200” in the patient whose clinical trial results are “OK”. In this example, there is a difference of four times between the patient whose clinical trial results are “OK” and the other patients, and it is assumed that a statistically significant difference is also identified. In this case, the update function 155 determines that the “tumor mutation burden” is the quantitative clinical information that can identify a significant difference in drug efficacy.

As a result of determination, there is quantitative clinical information that can identify a significant difference in drug efficacy, in the quantitative clinical information in the clinical trial with the clinical trial ID “C0001”. In this case, the update function 155 updates the conditions for administering the investigational drug used in the clinical trial with the clinical trial ID “C0001”, on the basis of the quantitative clinical information that can identify a significant difference in drug efficacy.

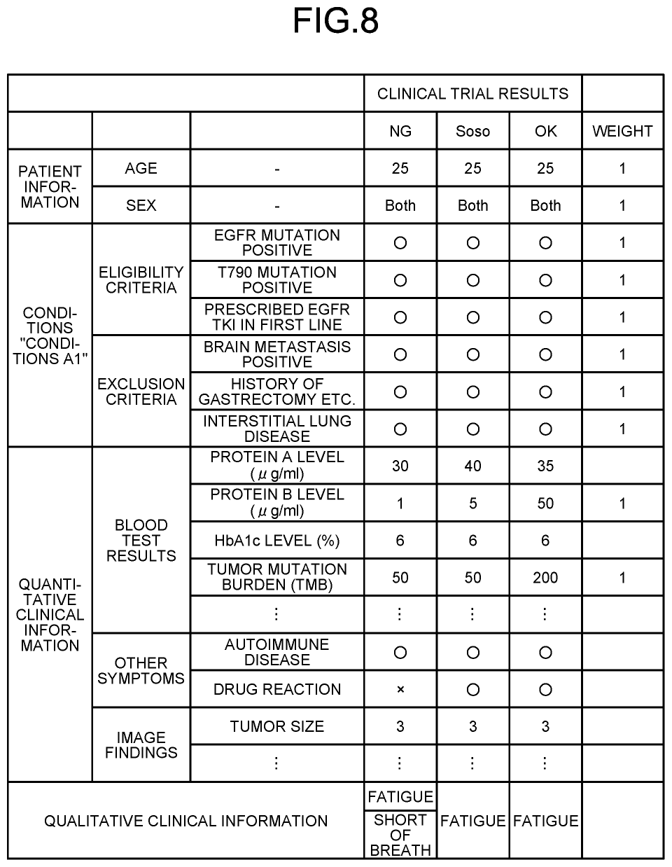

More specifically, the quantitative clinical information that can identify a significant difference in drug efficacy is the “protein B level” and the “tumor mutation burden”. In this case, as illustrated in FIG. 8, the update function 155 sets the weight “1” for the quantitative clinical information “protein B level” and “tumor mutation burden”, as the conditions for administering the investigational drug used in the clinical trial with the clinical trial ID “C0001”. In other words, the update function 155 updates the conditions for administering the investigational drug used in the clinical trial with the clinical trial ID “C0001”, by adding the quantitative clinical information “protein B level” and “tumor mutation burden” in the conditions.

In this example, the update function 155 sets “1” as the weight for the determined quantitative clinical information “protein B level” and “tumor mutation burden”. However, it is not limited thereto. For example, the update function 155 may also perform a statistically significant difference test, and set the weight according to the reliability of the significant difference in drug efficacy. For example, the update function 155 may calculate the reliability of the determined quantitative clinical information “protein B level” and “tumor mutation burden”, and when the obtained reliability indicates a significant difference in drug efficacy within a range of 90 and 100%, the update function 155 sets “1” as the weight. For example, when the obtained reliability indicates a significant difference in drug efficacy within a range of 75 and 90%, the update function 155 sets “0.8” as the weight. For example, when the obtained reliability indicates a significant difference in drug efficacy within a range of 50 and 75%, the update function 155 sets “0.5” as the weight. In this manner, the update function 155 updates the conditions for administering the investigational drug used in the clinical trial with the clinical trial ID “C0001”, by adding the quantitative clinical information “protein B level” and “tumor mutation burden” in the conditions.

Next, a process performed after the conditions for administering the investigational drug used in the clinical trial with the clinical trial ID “C0001” are updated will be described.

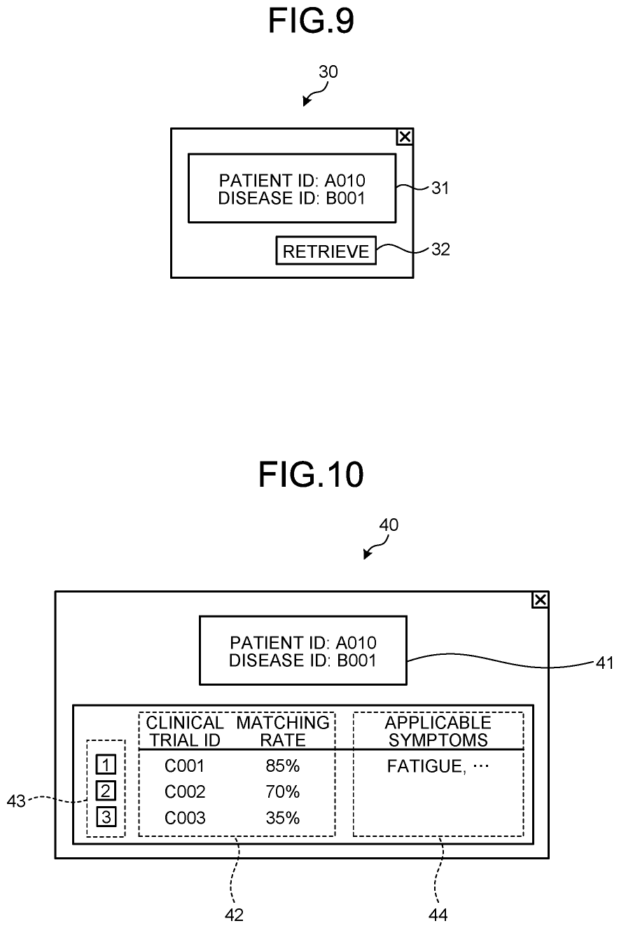

For example, the reception function 151 receives the patient information that is uploaded to the clinical trial matching apparatus 100 from the terminal 300 in the hospital, and that includes the patient ID “A010”. The reception function 151 then stores the received patient information in the patient information table 141 in the storage circuitry 140. In this case, the output processing function 153 transmits a message indicating that the patient information including the patient ID “A010” is received as the reception information, to the terminal 300 in the hospital. The terminal 300 receives the reception information, and causes the display of the terminal 300 to display a reception screen 30 illustrated in FIG. 9, as the received reception information. For example, the reception screen 30 includes a display field 31 that displays the patient ID “A010” and the disease ID “B001”, and a retrieval button 32 operated by the staff of the hospital to perform a retrieval request, which will be described below.

For example, it is assumed that when the reception screen 30 is displayed on the display of the terminal 300 in the hospital, the staff of the hospital operates the retrieval button 32 on the reception screen 30. In this case, the terminal 300 transmits a retrieval request to the clinical trial matching apparatus 100. The retrieval function 152 retrieves the clinical trial information that matches the patient information including the patient ID “A010”, from a plurality of pieces of clinical trial information stored in the clinical trial information table 142 in the storage circuitry 140, according to the retrieval request. In this process, the output processing function 153 transmits the retrieved clinical trial information to the terminal 300 in the hospital. The terminal 300 receives the clinical trial information, and causes the display of the terminal 300 to display a retrieval result screen 40 illustrated in FIG. 10, as the received clinical trial information. For example, the retrieval result screen 40 includes a display field 41 that displays the patient ID “A010” and the disease ID “B001”, a display field 42 that displays the retrieved clinical trial information, a selection button 43 that selects one clinical trial information from the retrieved clinical trial information, and a display field 44 that displays qualitative clinical information (information on QOL) as accessory information of the retrieved clinical trial information.

For example, it is assumed that the pieces of clinical trial information including the clinical trial IDs “C0001”, “C0002”, and “C0003” are retrieved by the retrieval function 152, as the clinical trial information that matches the patient information including the patient ID “A010”. For example, in the clinical trial with the clinical trial ID “C0001”, it is assumed that the conditions for administering the investigational drug used in the clinical trial (“eligibility criteria”, “exclusion criteria”, “protein B level”, and “tumor mutation burden”) match with the patient information including the patient ID “A010” at a high probability. In other words, the updated conditions match with the patient information including the patient ID “A010” at a high probability. In this process, it is assumed that the matching rates of the pieces of clinical trial information with respect to the clinical trial IDs “C0001”, “C0002”, and “C0003” are “85%”, “70%”, and “35%”, respectively. In this case, in the display of the terminal 300 in the hospital, the pieces of clinical trial information including the clinical trial IDs “C0001”, “C0002”, and “C0003” are displayed on the display field 42 of the retrieval result screen 40, in descending order of the matching rate. In other words, the matching rates of “85%”, “70%”, and “35%” are displayed on the display field 42 with respect to the clinical trial IDs “C0001”, “C0002”, and “C0003”, respectively. Moreover, the selection buttons 23 “1”, “2”, and “3” are also displayed on the display field 42 in descending order of the matching rate, with respect to the clinical trial IDs “C0001”, “C0002”, and “C0003”, respectively.

In the clinical trial with the clinical trial ID “C0001”, it is assumed that symptoms such as “fatigue” caused by the prescribed drug are indicated. In this case, in the display of the terminal 300 in the hospital, symptoms such as “fatigue” are displayed on the display field 44 of the retrieval result screen 40 with respect to the clinical trial ID “C0001”.

As described above, among the clinical trials with the clinical trial IDs “C0001”, “C0002”, and “C0003”, the clinical trial that matches the most with the patient information including the patient ID “A010” is the clinical trial with the clinical trial ID “C0001”. In this example, the patient with the patient ID “A010” may wish to participate in the clinical trial that matches the most with the patient, in expectation of significant drug efficacy. On the other hand, the patient with the patient ID “A010” may wish to participate in the clinical trial that matches the second with the patient, in expectation of drug efficacy but by avoiding symptoms such as “fatigue”.

First, a case in which the patient with the patient ID “A010” wishes to participate in the clinical trial that matches the most with the patient, in expectation of significant drug efficacy will be described.

For example, it is assumed that when the retrieval result screen 40 is displayed on the display of the terminal 300 in the hospital, the staff of the hospital operates the selection button 23 “1” on the retrieval result screen 40, and selects the clinical trial information including the clinical trial ID “C0001”, after consulting with the patient with the patient ID “A010”. In this case, the terminal 300 transmits a message indicating that the clinical trial information including the clinical trial ID “C0001” is selected to the clinical trial matching apparatus 100, as the selection information. According to the selection information, the output processing function 153 notifies the patient information such as the patient ID “A010” to the terminal 200 in the pharmaceutical company that has uploaded the clinical trial information including the clinical trial ID “C0001” to the clinical trial matching apparatus 100. In other words, the output processing function 153 introduces the patient with the patient ID “A010” to the pharmaceutical company as a clinical trial subject. In the hospital, the clinical trial with the clinical trial ID “C0001” is conducted on the patient with the patient ID “A010”, as the selected clinical trial information. In this manner, in the present embodiment, by feeding back the clinical trial results and the clinical information on the patient to the clinical trial matching apparatus 100, it is possible to more suitably match the patient with the clinical trial. As a result, it is possible to improve the results of clinical trials, because the clinical trials can be conducted by recruiting more stratified patients on the basis of clinical trial results.

Next, a case in which the patient with the patient ID “A010” wishes to participate in the clinical trial that matches the second with the patient in expectation of significant drug efficacy, but by avoiding symptoms such as “fatigue”, will be described.

For example, it is assumed that when the retrieval result screen 40 is displayed on the display of the terminal 300 in the hospital, the staff of the hospital operates the selection button 23 “2” on the retrieval result screen 40, and selects the clinical trial information including the clinical trial ID “C0002”, after consulting with the patient with the patient ID “A010”. In this case, the terminal 300 transmits a message indicating that the clinical trial information including the clinical trial ID “C0002” is selected, to the clinical trial matching apparatus 100, as the selection information. The output processing function 153 notifies the patient information such as the patient ID “A010” to the terminal 200 in the pharmaceutical company that has uploaded the clinical trial information including the clinical trial ID “C0002” to the clinical trial matching apparatus 100, according to the selection information. In other words, the output processing function 153 introduces the patient with the patient ID “A010” to the pharmaceutical company as a clinical trial subject. Moreover, in the hospital, the clinical trial with the clinical trial ID “C0002” is conducted on the patient with the patient ID “A010” as the selected clinical trial information. In this manner, in the present embodiment, by feeding back the clinical trial results and the clinical information on the patient to the clinical trial matching apparatus 100, the patient can select a desirable clinical trial. As a result, it is possible to improve the results of clinical trials, because the clinical trial suitable for the patient can be conducted.

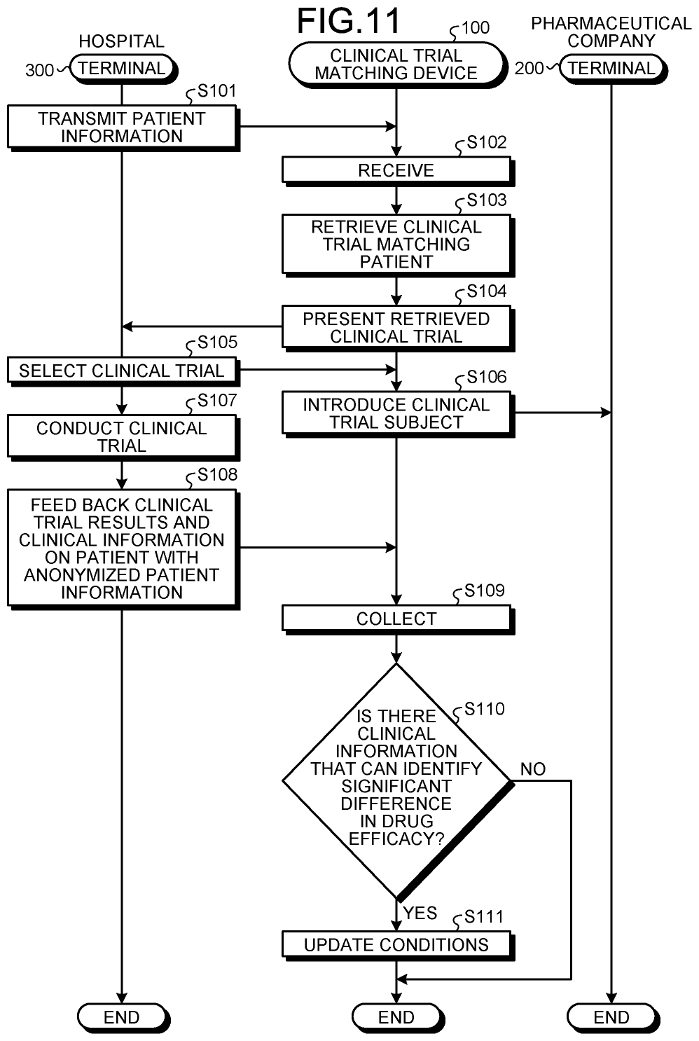

FIG. 11 is a flowchart illustrating a procedure of a process performed by the clinical trial matching system 1 including the clinical trial matching apparatus 100 according to the present embodiment.

The information on the patient (patient information) is transmitted to the clinical trial matching apparatus 100 from the terminal 300 in the hospital (step S101). The reception function 151 of the clinical trial matching apparatus 100 receives the patient information (step S102).

Next, the retrieval function 152 of the clinical trial matching apparatus 100 retrieves the clinical trial that matches the patient whose patient information is received, from a plurality of clinical trials in which conditions for administering the investigational drug are set, by referring to the clinical trial information table 142 in the storage circuitry 140 (step S103). In this process, the output processing function 153 presents the retrieved clinical trials to the terminal 300 in the hospital (step S104). The pieces of information on the retrieved clinical trials are displayed on the terminal 300 in the hospital in descending order of the matching rate.

The terminal 300 in the hospital selects one clinical trial from the presented clinical trials (step S105). In this process, the output processing function 153 of the clinical trial matching apparatus 100 notifies the patient information to the terminal 200 in the pharmaceutical company that has uploaded the information on the selected clinical trial. In other words, the output processing function 153 introduces the patient whose patient information is received, to the pharmaceutical company as a clinical trial subject (step S106).

The hospital that has selected the clinical trial conducts the clinical trial on the patient (step S107). The results of the conducted clinical trial and the clinical information on the patient are fed back to the clinical trial matching apparatus 100 from the terminal 300 in the hospital, with the anonymized patient information (step S108). The collection function 154 of the clinical trial matching apparatus 100 collects the clinical trial results and the clinical information on the patient from the terminal 300 in the hospital with the anonymized patient information. The collection function 154 then stores the results and information in the clinical trial information table 142 in the storage circuitry 140 (step S109).

The update function 155 of the clinical trial matching apparatus 100 determines whether there is clinical information that can identify a significant difference in drug efficacy, in the clinical information on the patient (step S110). When the update function 155 determines that there is clinical information that can identify a significant difference in drug efficacy (Yes at step S110), the update function 155 updates the conditions for administering the investigational drug used in the clinical trial with the clinical trial ID “C0001”, on the basis of the clinical information (step S111). On the other hand, when the update function 155 determines that there is no clinical information that can identify a significant difference in drug efficacy (No at step S110), step S111 is skipped and the process is completed.

In this manner, as described above, in the clinical trial matching apparatus 100 according to the present embodiment, the reception function 151 receives the information on the patient (patient information). The retrieval function 152 retrieves the clinical trial that matches the patient whose patient information is received from a plurality of clinical trials, by referring to the storage circuitry 140 that stores therein data for associating the clinical trial in which medication conditions are set with the results of the clinical trial, for the clinical trials. The output processing function 153 presents information on the retrieved clinical trial. The collection function 154 collects the clinical trial results obtained by conducting the retrieved clinical trial on the patient whose patient information is received, and the clinical information on the patient. The collection function 154 then stores the results and information in the storage circuitry 140. When there is clinical information that can identify a significant difference in drug efficacy in the collected clinical information, the update function 155 updates the above-described conditions of the retrieved clinical trial, on the basis of the clinical information. In this manner, in the present embodiment, by feeding back the clinical trial results and the clinical information on the patient to the clinical trial matching apparatus 100, it is possible to more suitably match the patient with the clinical trial. As a result, it is possible to improve the results of clinical trials, because the clinical trial suitable for the patient can be conducted.

Other Modifications

While the present embodiment has been described above, the embodiment may also be implemented in various different forms in addition to the embodiment described above.

In the present embodiment, the process executed by each function is described using the “lung cancer” as an example of a disease. However, it is not limited thereto, and the similar process may also be performed on other diseases.

In the present embodiment, for example, the process executed by the update function 155 is executed every time the collection function 154 collects the clinical trial results and the clinical information on the patient, when the number of results of the clinical trial with the clinical trial ID “C0001” exceeds a set number. However, it is not limited thereto. For example, as a modification of the present embodiment, the process executed by the update function 155 may also be performed regularly at every set period of time, when the number of results of the clinical trial with the clinical trial ID “C0001” exceeds a set number.

In the present embodiment, for example, the clinical trial matching apparatus 100 is used to find a clinical trial suitable for the patient. However, it is not limited thereto. For example, as a modification of the present embodiment, the clinical trial matching apparatus 100 may also be used to find a patient suitable for the clinical trial.

For example, the reception function 151 receives the information on the clinical trial to be conducted (clinical trial information). The retrieval function 152 retrieves a patient matching the clinical trial to be conducted from a plurality of patients on the basis of the clinical trial information, by referring to the storage circuitry 140 that stores therein data for associating the clinical trial in which medication conditions are set with the results of the clinical trial, for the clinical trials. The output processing function 153 presents information on the retrieved patient. The collection function 154 collects the clinical trial results obtained by conducting the clinical trial to be conducted on the retrieved patient and the clinical information on the patient. The collection function 154 then stores the results and information in the storage circuitry 140. When there is clinical information that can identify a significant difference in drug efficacy in the collected clinical trial information, the update function 155 updates the above-described conditions of the clinical trial to be conducted, on the basis of the clinical information. In this case also, in the modification of the present embodiment, by feeding back the clinical trial results and the clinical information on the patient to the clinical trial matching apparatus 100, it is possible to more suitably match the patient with the clinical trial. As a result, it is possible to improve the results of clinical trials.

The components of the devices according to the embodiment described above are functionally conceptual, and need not necessarily be physically configured as illustrated. In other words, the specific modes of dispersion and integration of the devices are not limited to those illustrated in the drawings, and all or some of the devices can be configured in a functionally or physically dispersed or integrated manner in any units according to various types of loads or use conditions. Moreover, all or any part of the processing functions performed by the devices can be implemented by a CPU or a computer program analyzed and executed by the CPU, or can be implemented as hardware based on a wired logic.

The methods explained in the embodiment described above may be implemented by executing a prepared control program on a computer such as a personal computer and a work station. The control program may be distributed via a network such as the Internet. The control program may also be recorded in a computer-readable recording medium such as a hard disk, a flexible disk (FD), a compact disc-read only memory (CD-ROM), magneto optical (MO), and a digital versatile disc (DVD), and executed by being read out from the storage medium by the computer.

According to at least one embodiment described above, it is possible to improve the results of clinical trials. Moreover, in addition to the clinical trial, the embodiment can also be used for evaluating the drug efficacy of existing therapeutic agents, and optimizing the usage and dosage of therapeutic agents.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A clinical trial matching apparatus, comprising processing circuitry configured:
to receive information on a patient;
to retrieve a clinical trial that matches the patient from a plurality of clinical trials based on the information on the patient, by referring to storage circuitry that stores therein data for associating a clinical trial in which a medication condition is set with a result of the clinical trial, for the clinical trials;
to present information on the retrieved clinical trial;
to collect a clinical trial result obtained by conducting the retrieved clinical trial on the patient having the received information, and clinical information on the patient, and to store the clinical trial result and the clinical information in the storage circuitry; and
to update the condition of the retrieved clinical trial based on the clinical information, when there is clinical information that enables identification of a significant difference in drug efficacy in the collected clinical information.

2. The clinical trial matching apparatus according to claim 1, wherein
the processing circuitry retrieves the clinical trials that match the patient by referring to the storage circuitry, and
the processing circuitry presents a screen used to select one clinical trial from the retrieved clinical trials, and information on quality of life (QOL) as accessory information of the clinical trial.

3. The clinical trial matching apparatus according to claim 2, wherein the processing circuitry collects clinical information including the information on QOL.

4. The clinical trial matching apparatus according to claim 1, wherein the processing circuitry collects clinical information including information on genes.

5. The clinical trial matching apparatus according to claim 2, wherein the processing circuitry collects clinical information including information on genes.

6. The clinical trial matching apparatus according to claim 3, wherein the processing circuitry collects clinical information including information on genes.

7. The clinical trial matching apparatus according to claim 1, wherein the processing circuitry collects anonymized clinical information.

8. The clinical trial matching apparatus according to claim 2, wherein the processing circuitry collects anonymized clinical information.

9. The clinical trial matching apparatus according to claim 3, wherein the processing circuitry collects anonymized clinical information.

10. The clinical trial matching apparatus according to claim 4, wherein the processing circuitry collects anonymized clinical information.

11. The clinical trial matching apparatus according to claim 1, wherein the condition includes an eligibility criterion for assessing eligibility for a clinical trial, and an exclusion criterion that does not meet the eligibility criterion.

12. The clinical trial matching apparatus according to claim 2, wherein the condition includes an eligibility criterion for assessing eligibility for a clinical trial, and an exclusion criterion that does not meet the eligibility criterion.

13. The clinical trial matching apparatus according to claim 3, wherein the condition includes an eligibility criterion for assessing eligibility for a clinical trial, and an exclusion criterion that does not meet the eligibility criterion.

14. The clinical trial matching apparatus according to claim 4, wherein the condition includes an eligibility criterion for assessing eligibility for a clinical trial, and an exclusion criterion that does not meet the eligibility criterion.

15. The clinical trial matching apparatus according to claim 5, wherein the condition includes an eligibility criterion for assessing eligibility for a clinical trial, and an exclusion criterion that does not meet the eligibility criterion.

16. A clinical trial matching apparatus, comprising processing circuitry configured:
to receive information on a clinical trial to be conducted;
to retrieve a patient matching the clinical trial to be conducted from a plurality of patients based on the information on the clinical trial to be conducted, by referring to storage circuitry that stores therein data for associating a clinical trial in which a medication condition is set with a result of the clinical trial, for a plurality of the clinical trials;
to present information on the retrieved patient;
to collect the clinical trial result obtained by conducting the clinical trial to be conducted on the retrieved patient and clinical information on the patient, and store the clinical trial result and the clinical information in the storage circuitry; and
to update the condition of the clinical trial to be conducted based on the clinical information, when there is clinical information that enables identification of a significant difference in drug efficacy in the collected clinical information.
